(19) United States
(12) Patent Application Publication
(10) Pub. No.: US 2025/0144292 A1
(43) Pub. Date: May 8, 2025

(54) SYSTEMS AND METHODS FOR INTERNAL DRUG LOADING FOR INFUSION PROCESSES

(71) Applicant: Eli Lilly and Company, Indianapolis, IN (US)

(72) Inventors: Yun-Chu CHEN, West Lafayette, IN (US); Sarena Delvecchio HORAVA, Cambridge, MA (US); Coralie Adele RICHARD, Indianapolis, IN (US); Monica Rixman SWINNEY, Stoneham, MA (US); Yoon YEO, West Lafayette, IN (US)

(21) Appl. No.: 18/835,272

(22) PCT Filed: Feb. 15, 2023
(86) PCT No.: PCT/US2023/013072

§ 371 Date: Aug. 1, 2024

Related U.S. Application Data
(60) Provisional application No. 63/311,647, filed on Feb. 18, 2022.

(51) Int. Cl. A61M 5/158; A61K 31/5415; A61L 29/08; A61L 29/16; A61M 5/142

(52) U.S. Cl. CPC A61M 5/158; A61K 31/5415; A61L 29/085; A61L 29/16; A61M 5/14248; A61L 2300/41; A61L 2300/606; A61L 2300/63; A61L 2420/02; A61M 2205/0238; A61M 2207/00

(57) ABSTRACT

Methods for applying a therapeutic agent coating to an extended-wear device and the systems created thereby are disclosed. For example, a method for coating an extended-wear device using N-TIPS provides therapeutic agent coating for localized treatment in a patient using an extended-wear device for monitoring or treatment of illness or injury, wherein such methods can be used on polymeric films or within polymeric tubes.

### FIELD OF THE DISCLOSURE

The present disclosure relates to systems and methods for loading of therapeutic agents within catheters and related tubing. In particular, the present disclosure relates to a drug-eluting surface for extended drug release to mitigate the damages of biofouling and inflammatory responses during use of implantable medical devices.

### BACKGROUND OF THE DISCLOSURE

Monitoring, control, and treatment of chronic disorders may include implantation of medical devices within the body. For example, a catheter may be provided for continuous infusion of a therapeutic agent into patients having a variety of injuries and/or diseases. Such catheter use may require extended wear by the patient, which may increase the risk of biofouling and inflammatory responses, further increasing the risk of device failure or otherwise limiting the applications of such devices.

As an example of extended wear use, continuous subcutaneous insulin infusion (“CSII”) may be performed using an insulin infusion set (“IIS”). One example of an IIS device 100 is shown in FIG. 1. The illustrative device 100 includes a first, proximal end 112 that communicates with an insulin reservoir of a pump (not shown) to receive an insulin formulation and a second, distal end 114 that communicates with a patient (not shown) to deliver the insulin formulation (i.e. the infusate). At the first end 112, the illustrative device 100 includes a reservoir connector 120 configured to couple with the insulin reservoir, a line set tubing 122, and a base connector 124. At the second end 114, the illustrative device 100 includes an infusion base 130 configured to receive the base connector 124, an adhesive pad 132 configured to adhere the infusion base 130 to the patient's skin, and an infusion catheter or needle 134 configured for insertion into the patient's skin. In use, the insulin formulation is directed from the pump, through the line set tubing 122, through the infusion catheter 134, and into the patient's subcutaneous (SC) tissue.

IIS devices may vary in size, shape, appearance, materials, and other features. In one example, the material used to construct the infusion catheter 134 may vary (e.g., the Contact Detach™ Infusion Set available from Animas Corporation uses a steel infusion catheter, whereas the MiniMed® Quick-set® Infusion Set available from Medtronic uses a plastic infusion catheter). In another example, the arrangement of line set tubing 122 may vary (e.g., the Contact Detach™ Infusion Set available from Animas Corporation uses two sets of a line set tubing coupled together via an intermediate strain-relief base, whereas the MiniMed® Quick-set® Infusion Set available from Medtronic uses a single line set tubing).

The patient's body may exhibit an inflammatory and/or foreign body response at the site of the infusion catheter 134. This response at the infusion site may vary from patient to patient depending on various factors, including the patient's susceptibility to wound formation, the patient's associated tissue remodeling and the patient's sensitivity to the particular insulin formulation, including phenolic excipients (e.g., m-cresol, phenol, methylparaben, ethylparaben, butylbaraben, other preservatives, and combinations thereof) in the insulin formulation, for example. M-cresol, for example, has been shown to induce inflammatory pathways, negatively impact human immune cell types in vitro, degrade lipid bilayers and neuronal cell membranes, and induce aggregation of proteins and initiate protein unfolding, each of which might contribute to infusion site events.

Due to these inflammatory and/or foreign body responses at the infusion sites, known IIS devices for CSII are currently indicated for two- to three-day (2-3 d) use. After even a short wear time, the inflammatory and/or foreign body response may impair the efficacy of the patient's infusion site, thereby limiting insulin uptake, increasing the risk of hyperglycemia, and limiting viable infusion site longevity. The limited wear time for IIS devices represents a two- to seven-times discrepancy compared with the wear time for continuous glucose monitors (CGMs), thus introducing an obstacle to achieving a convenient, fully integrated CSII/CGM artificial pancreas system.

Localized drug elution and introduction at the device site mitigates such issues and allows for a longer wear time, improving efficiency and lowering costs for both the patient and the treating practitioner. However, drug elution may further be accomplished by providing the therapeutic coating along the interior of a catheter or related tubing or on other surfaces of implanted devices or post-surgical wound dressings.

### SUMMARY

Methods for applying a therapeutic agent coating to an extended-wear device and the systems created thereby are disclosed. For example, a method for coating an extended-wear device using N-TIPS is disclosed to provide therapeutic agent coating for localized treatment in a patient using an extended-wear device for monitoring or treatment of illness or injury, wherein such methods can be used on polymeric films or within polymeric tubes.

In a first aspect of the present disclosure, a method of coating a medical device with a therapeutic agent is disclosed. The method comprises: introducing a solution comprising the therapeutic agent to a surface of the medical device; freezing the medical device with the solution so that the therapeutic agent precipitates from the solution; and washing the medical device with a non-solvent, wherein the precipitated therapeutic agent remains as a coating on the surface of the medical device.

In a second aspect of the present disclosure, an infusion device for extended wear is disclosed. The infusion device comprises: a base; an adhesive configured to couple the base to a skin of a patient; and a catheter configured to pierce the skin of the patient, wherein the catheter is in fluid communication with a tubing including a therapeutic coating on an inner surface of an inner lumen defined by the tubing, the therapeutic coating disposed on the inner surface of the inner lumen defined by the tubing using N-TIPS.

In various aspects of the present disclosure, the surface may be an inner surface of an inner lumen of a catheter. The catheter may be a component of an infusion device.

In various aspects of the present disclosure, the surface may be a surface of a film.

In various aspects of the present disclosure, the surface may be a polymeric surface. The surface may comprise low-density polyethylene.

In various aspects of the present disclosure, the method may further comprise the step of pretreating the surface of the medical device with at least one of a heated pretreatment or a plasma-etching pretreatment before introducing the solution comprising the therapeutic agent to the surface.

In various aspects of the present disclosure, the method further comprises the step of heating the medical device with the solution to a temperature of about 80° C. after introducing the solution to the surface.

In various aspects of the present disclosure, the therapeutic agent may crystallize on the surface during the freezing step.

In various aspects of the present disclosure, only the therapeutic agent may precipitate. The therapeutic agent may be water-insoluble. The therapeutic agent may be meloxicam.

In various aspects of the present disclosure, a matrix comprising the therapeutic agent and a polymer may precipitate. The therapeutic agent may be water-soluble.

In various aspects of the present disclosure, the therapeutic coating may consist essentially of a therapeutic agent. The therapeutic agent may be meloxicam.

In various aspects of the present disclosure, the catheter may be configured to remain inserted in the patient for 14 days, and the therapeutic coating may be configured to continuously release the therapeutic agent while the catheter is inserted.

In various aspects of the present disclosure, the therapeutic agent may be in crystalline form.

In various aspects of the present disclosure, the tubing may be comprised of low-density polyethylene.

### BRIEF DESCRIPTION OF THE DRAWINGS

The above-mentioned and other features and advantages of this disclosure, and the manner of attaining them, will become more apparent and will be better understood by reference to the following description of embodiments of the invention taken in conjunction with the accompanying drawings, wherein:

FIG. 1 is an illustration of a top plan view of a known insulin infusion set (“IIS”) device;

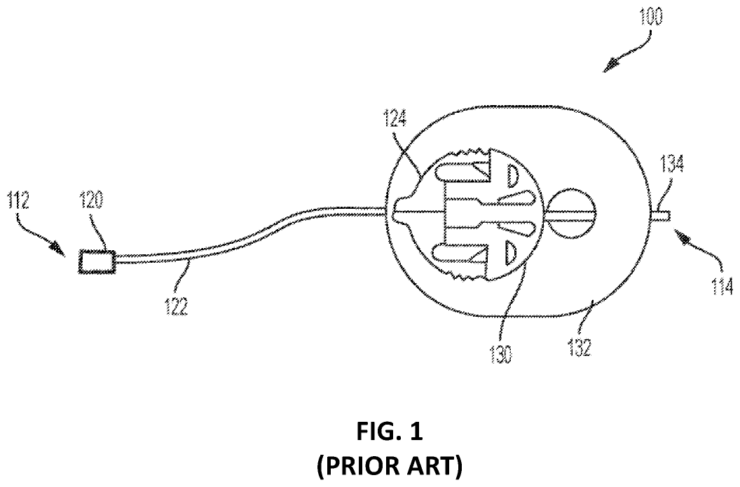

FIG. 2 is a cross-sectional view of an exemplary IIS device, the device including a reservoir connector, a line set tubing, a base connector, and an infusion base;

FIG. 3 is a flow diagram depicting a method for applying a therapeutic agent to a polymeric film;

FIG. 4 is a flow diagram depicting a method for applying a therapeutic agent to an inner surface of a hollow tube;

FIG. 5 is a graph illustrating a comparison of the average drug loading of polymeric films pretreated using differing processes and further comparing the average drug loading of polymeric films which were subjected to room temperature drug loading or high temperature drug loading;

FIG. 6 is a scanning electron microscopic (“SEM”) image illustrating a comparison of the microstructure of polymeric films pretreated using differing processes before the polymeric films include a drug loading;

FIG. 7 is an SEM image comparing increasingly magnified views of a drug loaded polymeric film, wherein the polymeric film unit was left untreated during a pretreatment phase and the polymeric film was heated to 80° C. during the drug loading process;

FIG. 8 is an SEM image comparing increasingly magnified views of a drug loaded polymeric film, wherein the polymeric film was subjected to heated pretreatment during the pretreatment phase and the polymeric film was heated to 80° C. during the drug loading process;

FIG. 9 is an SEM image comparing increasingly magnified views of a drug loaded polymeric film, wherein the polymeric film was subjected to plasma-etching pretreatment during the pretreatment phase and the polymeric film was heated to 80° C. during the drug loading process;

FIG. 10 is a graph illustrating in comparison the average drug loading of tubes pretreated using differing processes and further comparing the average drug loading of tubes which were subjected to room temperature drug loading or high temperature drug loading;

FIG. 11 is an SEM image showing the microstructure of a tube left untreated during the pretreatment phase before the tube includes a drug loading;

FIG. 12 is an SEM image showing the microstructure of a tube subjected to heated pretreatment during the pretreatment phase before the tube includes a drug loading;

FIG. 13 is an SEM image showing the microstructure of a tube subjected to plasma-etching during the pretreatment phase before the tube includes a drug loading;

FIG. 14 is an SEM image comparing increasingly magnified views of a drug loaded tube, wherein the tube was left untreated during the pretreatment phase;

FIG. 15 is an SEM image comparing increasingly magnified views of a drug loaded tube, wherein the tube was subjected to heated pretreatment during the pretreatment phase;

FIG. 16 is an SEM image comparing increasingly magnified views of a drug loaded tube, wherein the tube was subjected to plasma-etching during the pretreatment phase; and

FIG. 17 is a graph illustrating in comparison the average cumulative release % of a therapeutic agent over 14 days from tubes pretreated using differing processes.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

### Example Device

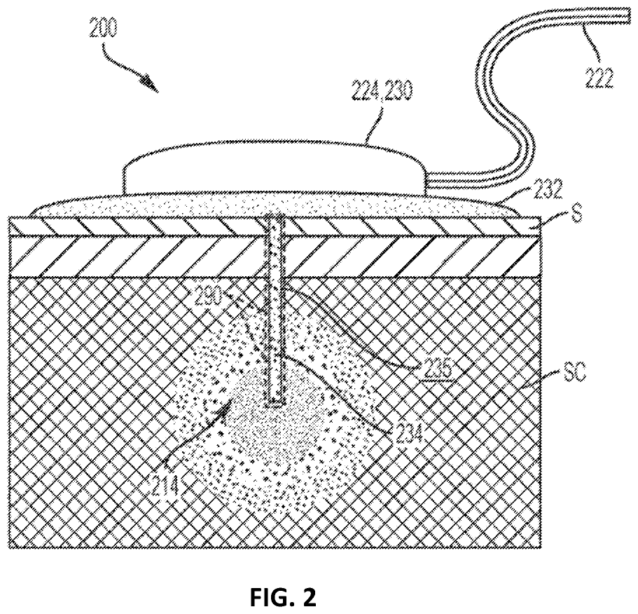

An example insulin infusion set device 200 is shown in FIG. 2. The device 200 includes a base connector 224 in the shape of a male buckle portion and an infusion base 230 in the shape of a female buckle portion configured to receive the base connector 224. An adhesive pad 232 is configured to adhere the infusion base 230 and the coupled base connector 224 to the patient's skin S. An infusion element in the form of an infusion catheter 234 is configured for insertion into the patient's subcutaneous SC tissue and is fluidly coupled to the infusion base 230 and the base connector 224 of the device. It is also within the scope of the present disclosure for the infusion element to be a needle. Flexible line set tubing 222 fluidly couples the infusion base 230 and the base connector 224 to a reservoir connector (not shown) that is configured to couple with an insulin reservoir (not shown). In use, the insulin formulation is directed from the pump, through the line set tubing 222, through the infusion catheter 234, and into the patient's subcutaneous SC tissue. Exemplary insulin formulations include, but are not limited to, insulin analogs such as insulin lispro or insulin glargine, insulin derivatives, and other insulins.

The infusion catheter 234 may be constructed of steel, plastic (e.g., polytetrafluoroethylene (PTFE), low-density polyethylene (LDPE), ethyl vinyl acetate (EVA), copolymers thereof, and combinations thereof), or another suitable material. The infusion catheter 234 may be sufficiently thick to withstand implantation while being sufficiently thin to promote patient comfort. In some embodiments, the infusion catheter 234 may have a thickness less than about 200 μm, less than about 150 μm, or less than about 100 μm, for example.

A therapeutic coating 290 may be configured to release and deliver one or more therapeutic agents to the patient in an extended manner, as described further below. The application of the therapeutic coating 290 to device 200 may vary. In certain embodiments, the therapeutic coating 290 may be incorporated (e.g., embedded) directly into device 200. In other embodiments, the therapeutic coating 290 may be applied (e.g., coated) onto an underlying surface of the device 200, such as an interior surface of the infusion catheter 234 or an interior surface of the line set tubing 222. In other embodiments, the therapeutic coating 290 may be applied onto a filtration mechanism that is loaded into the device 200.

The location of the therapeutic coating 290 on the device 200 may also vary. In the illustrated embodiment of FIG. 2, for example, the therapeutic coating 290 is coated onto an outer surface 235 of the infusion catheter 234. In other embodiments, and as noted above and described further herein, the therapeutic coating 290 may be incorporated within, coated upon an inside surface of, or otherwise positioned within the infusion catheter 234. In this embodiment, the infusion catheter 234 and the therapeutic coating 290 may or may not be integrally formed of the same material. In some embodiments, the therapeutic coating 290 is located along the fluid pathway of the device 200. More specifically, in some embodiments, the therapeutic coating 290 is located inside the line set tubing 222, inside the base connector 224, inside the infusion base 230, and/or inside the infusion catheter 234 such that the therapeutic coating 290 may dissolve into the insulin formulation traveling through device 200 for simultaneous delivery to the patient. In some embodiments, the therapeutic coating 290 may be applied to only a portion of the interior length of the infusion catheter 234, the line set tubing 222, or other tubing as discussed herein. In other embodiments, the therapeutic coating 290 may be applied to the entirety of the interior length of the infusion catheter 234, the line set tubing 222, or other tubing as discussed herein. In embodiments in which the therapeutic coating 290 is only applied to a portion of the interior length of the tubing the therapeutic coating 290 may be applied, for example, to a portion of the interior length adjacent to the base connector 224 and/or infusion base 230 to allow for dissolution of the therapeutic coating 290 within a portion of the fluid path which is near the fluid path exit of device 200.

The device 200 may include various other features designed to achieve longevity in CSII infusion site viability. As a result, the infusion site may last longer than 3 days, 5 days, 7 days, or more, such as about 7 to 14 days, which may reduce insulin waste, reduce scarring, and enable a once-weekly or once-biweekly change-over time frame for a fully integrated artificial pancreas system. Further features of infusion sets and greater descriptions of features may be found in U.S. Patent Application Publication No. 2019/0054233 to DEMARIA, et al., published Feb. 21, 2019, and titled “INFUSION SET WITH COMPONENTS COMPRISING A POLYMERIC SORBENT TO REDUCE THE CONCENTRATION OF M-CRESOL IN INSULIN”, the disclosure of which is hereby expressly incorporated by reference in its entirety.

It is understood that although the disclosure herein is described in terms of IIS devices and infusion catheters, the therapeutic coating described herein may also be applied to other medical devices, including other catheters, pacemakers, heart valves, stents, and biosensors using the methods described herein. In other words, it is within the scope of the disclosure to apply a therapeutic coating using the methods described herein to a variety of implantable or other long-term contact medical devices.

### Therapeutic Agents

The therapeutic coating described herein may include one or more therapeutic agents in the form of anti-inflammatory agents, including nonsteroidal anti-inflammatory therapeutic agents (NSAIDs). Exemplary anti-inflammatory agents include meloxicam, bromfenac, ibuprofen, naproxen, aspirin, plumbagin, plumericin, celecoxib, diclofenac, etodolac, indomethacin, ketoprofen, ketorolac, nabumetone, oxaprozin, piroxicam, salsalate, sulindac, tolmetin, rapamycin, dexamethasone, betamethasone, heparin, sirolimus, and paxlitaxel, for example. A device site, and its corresponding device, may last longer when a NSAID is locally administered, resulting in further benefits for the patient, including use of fewer devices, fewer needle sticks, and avoidance of hyperglycemia that is associated with an inflammatory response. Controlled release of a NSAID locally at the insertion site may allow the device site and its corresponding device to last for an extended time period longer than 3 days, 5 days, 7 days, or more, such as about 7 to 14 days.

### Application of Therapeutic Agents to Devices

Phase separation techniques are often used in the creation of polymeric membranes or tissue scaffolds. Phase separation includes processes in which a homogenous system of two components is separated into its respective components via precipitation or solidification. Phase separation is commonly used for separation of homogenous solutions including a drug and a solvent or a polymer and a solvent. Two common phase separation processes include thermally induced phase separation (“TIPS”) and non-solvent induced phase separation (“NIPS”).

Conventionally, TIPS includes the steps of (i) dissolving a polymer in a high-boiling, low molecular weight solvent; (ii) casting the solution into a desired shape (e.g., flat sheet, fiber, etc.); (iii) cooling the solution at a low temperature in a controlled manner to induce precipitation of the polymer (phase separation); and (iv) removing the solvent by flushing with another medium (liquid or gas) to yield a polymer membrane. NIPS includes the steps of (i) dissolving a polymer in a high-boiling, low molecular weight solvent; (ii) casting the solution into a desired shape (e.g., flat sheet, fiber, etc.); and (iii) immersing the solution into a non-solvent bath to induce phase separation and extract the solvent. TIPS and NIPS may be carried out simultaneously, where the medium used in step (iv) of the TIPS process is miscible with the solvent and configured to serve as a non-solvent of the polymer. Such combined process is referred to as “N-TIPS”, as discussed further herein.

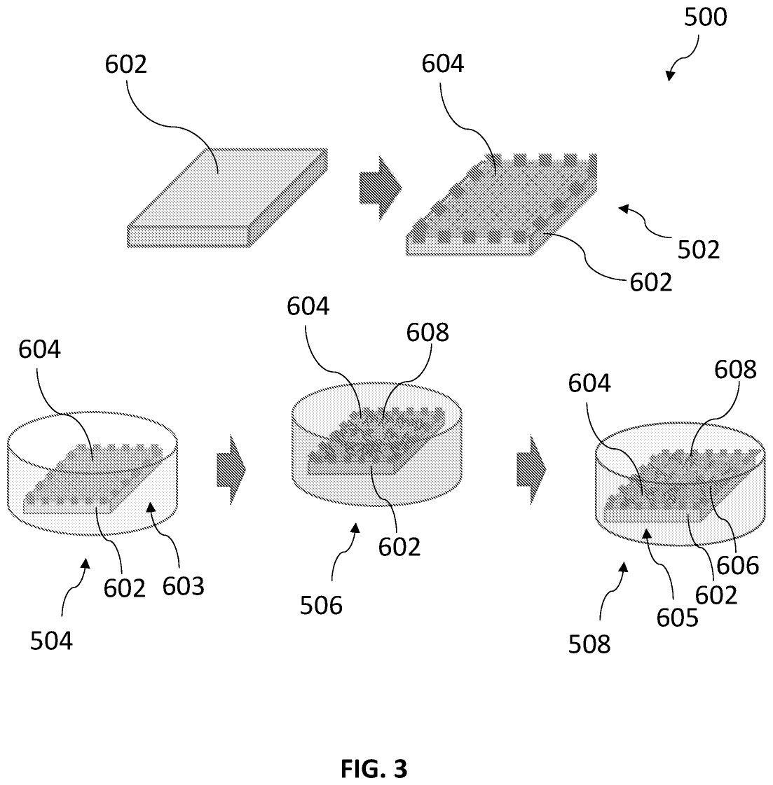

According to the present disclosure, as opposed to forming polymer films and tubes, such polymer films and tubes may be coated with a therapeutic agent using the N-TIPS method provided above. Referring to FIG. 3, a method 500 of loading a therapeutic agent on a polymeric film 602 is disclosed. The polymeric film 602 may be subjected to a pretreatment process at step 502, which may create a rough surface 604 on the polymeric film 602. In some embodiments, the polymeric film 602 may be left untreated. In other embodiments, the polymeric film 602 may be exposed to a high temperature for heated pretreatment. The high temperature may be at least, for example, about 75° C., about 80° C., about 85° C., about 90° C., or about 95° C. In other embodiments, the pretreatment temperature may be any temperature which is below the melting temperature and above the glass transition temperature of the underlying polymeric film 602. Varying pretreatment processes may result in differential changes in the surface morphology of the polymeric film 602 by, for example, creating relatively rougher or less rough surfaces in comparison with other pretreatment processes. As an example, treating the polymeric film 602 with plasma etching as described further herein may result in a rougher surface 604 than heated pretreatment processes or leaving the polymeric film 602 untreated as further illustrated below. An example of such plasma etching process is further described in U.S. Application No. 63/188,615, file don May 14, 2021, and titled “SYSTEMS AND METHODS FOR IGNITING PLASMA WITHIN TUBES”, the disclosure of which is hereby incorporated by reference. The capability of varying the surface roughness of the polymeric film 602 may be desirable for the precipitation of the desired therapeutic agent, as described further herein, as a greater surface roughness may provide a greater surface area on which the therapeutic agent may crystallize or otherwise precipitate.

The pretreated or untreated polymeric film 602 is then dipped in a therapeutic agent solution 603 at step 504. The therapeutic agent solution 603 may be comprised of a solvent and a water-insoluble therapeutic agent. In other embodiments, the therapeutic agent solution 603 may be comprised of a solvent, a water-soluble therapeutic agent, and a polymer. In yet other embodiments, the therapeutic agent solution 603 may be comprised of a solvent, a water-insoluble therapeutic agent, and an optional polymer. In some embodiments, the solvent may be dimethyl sulfoxide benzyl alcohol (DMSO-Bn). In the same or in other embodiments, the therapeutic agent may be meloxicam.

The concentration of the therapeutic agent solution 603 may be from about 10 mg/mL to about 40 mg/mL. In some embodiments, for example, the concentration of the therapeutic agent solution 603 may be 15 mg/mL. In yet other embodiments, the concentration of the therapeutic agent solution 603 may be 30 mg/mL. Step 504 may be fully completed at room temperature, or about 20° C., in some embodiments. In other embodiments, the loaded polymeric film 602 may be heated to 80° C. for at least one hour to provide a high temperature drug loading process. Such heated process may increase the drug loading amount as a result of decreased interfacial tension between the drug solution and the surface of the polymeric film 602.

The loaded polymeric film 602 is then frozen at step 506 to induce phase separation via TIPS as described above. In embodiments including water-soluble therapeutic agents, a polymer 608 and therapeutic agent 606 matrix precipitates during the TIPS process. In embodiments including water-insoluble therapeutic agents, the therapeutic agent 606 may precipitate alone during the TIPS process. In such embodiments, the therapeutic agent 606 may form crystals on the polymeric film 602. At step 508, a solvent exchange occurs, in which water 605 is washed over the polymeric film 602, removing the solvent from the polymeric film 602 and leaving behind the therapeutic agent 606 or the polymer 608 and therapeutic agent 606 matrix. In some embodiments, the water may be acidic. In other embodiments, the water may be basic or neutral. The acidity of the water used may depend on the therapeutic agent desired. For example, acidic water may be used for meloxicam. The polymeric film 602 may then be applied to a device as described above for extended-wear localized drug elution to provide the benefits discussed above.

The polymeric film 602 may comprise any suitable polymer for carrying out the described method. For example, the polymeric film 602 may comprise a polymer substantially unaffected during processing of the therapeutic coating. In some embodiments, the polymeric film 602 may comprise, for example, low-density polyethylene (LDPE). In other embodiments, polymeric film 602 may comprise at least one of, for example, thermoplastic polyurethane (TPU), polytetrafluoroethylene (PTFE), polyethylene (including high-density polyethylene (HDPE)), polycaprolactone (PCL), silicone, and poly-lactic-acid (PLA), as well as other suitable polymers.

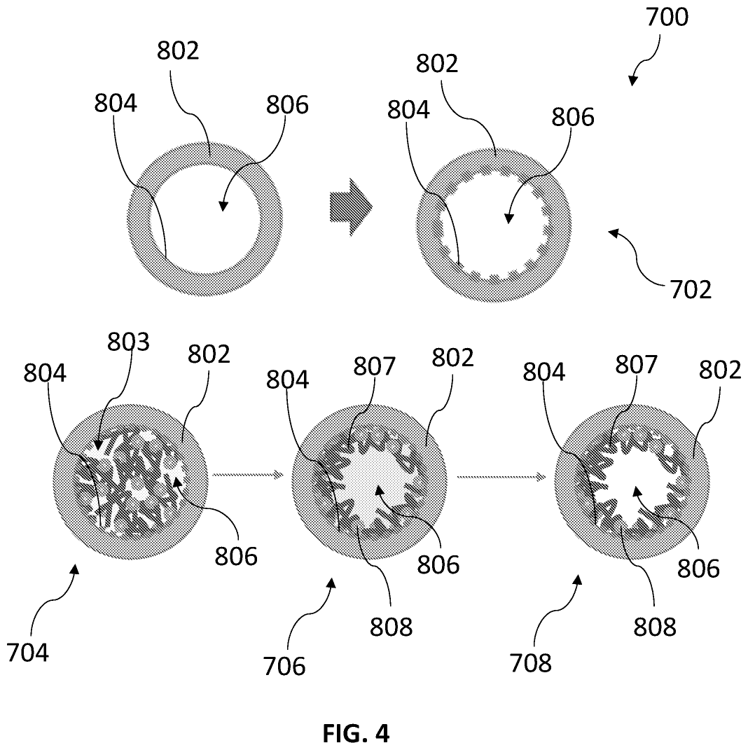

Now referring to FIG. 4, a method 700 is disclosed. Method 700 is similar to method 500 described above, except that method 700 is provided for the application of a therapeutic coating to an inner surface 804 of a hollow tube 802. For example, hollow tube 802 may be comprised of a catheter tube. Hollow tube 802 defines an inner lumen 806 defining an inner surface 804 of the hollow tube 802.

Like method 500 above, hollow tube 802 may be subjected to a pretreatment process at step 702, which may provide for a rougher inner surface 804 as described above. In some embodiments, hollow tube 802 may be left untreated. In other embodiments, hollow tube 802 may be exposed to a high temperature for heated pretreatment. The high temperature may be at least, for example, about 75° C., about 80° C., about 85° C., about 90° C., or about 95° C. In other embodiments, the pretreatment temperature may be any temperature which is below the melting temperature and above the glass transition temperature of the underlying hollow tube 802. As described above, varying pretreatment processes may result in differential changes in the surface morphology of the inner surface 804 of the hollow tube 802.

A syringe (not shown) is then used to introduce a therapeutic agent solution 803 to the inner lumen 806 of the hollow tube 802 at step 704. As described above, the therapeutic agent solution 803 may be comprised of a solvent and a water-insoluble therapeutic agent. In other embodiments, the therapeutic agent solution 803 may be comprised of a solvent, water-soluble therapeutic agent, and a polymer. In yet other embodiments, the therapeutic agent solution 803 may be comprised of a solvent, a water-insoluble therapeutic agent, and an optional polymer. In some embodiments, the solvent may be dimethyl sulfoxide benzyl alcohol (DMSO-Bn). In the same or in other embodiments, the therapeutic agent may be meloxicam.

As described above, the concentration of the therapeutic agent solution 803 may be from about 10 mg/mL to about 40 mg/mL. Step 704 may be fully completed at room temperature, or about 20° C., in some embodiments. In other embodiments, the loaded hollow tube 802 may be heated to 80° C. for at least one hour to provide a high temperature drug loading process as described above.

The loaded hollow tube 802 is then frozen at step 706 to induce phase separation via TIPS as described above. In embodiments including water-soluble therapeutic agents, a polymer 807 and therapeutic agent 808 matrix precipitates during the TIPS process. In embodiments including water-insoluble therapeutic agents, the therapeutic agent 808 precipitate may precipitate alone during the TIPS process. In such embodiments, the therapeutic agent 808 may form crystals on the inner surface 804 of the hollow tube 802. At step 708, a solvent exchange occurs, in which water is introduced into the inner lumen 806 of the hollow tube 802 via, for example, a syringe, removing the solvent from the hollow tube 802 and leaving behind the therapeutic agent 808 or the polymer 807 and therapeutic agent 808 matrix. In some embodiments, the water may be acidic. In other embodiments, the water may be basic or neutral. The acidity of the water used may depend on the therapeutic agent desired. For example, acidic water may be used for meloxicam. The hollow tube 802 may then be utilized as a medical device, i.e. a catheter, to introduce insulin or other therapeutic agents. During such introduction, the therapeutic coating may elute for localized treatment of the device site.

The hollow tube 802 may comprise any suitable polymer for carrying out the described method. For example, the hollow tube 802 may comprise a polymer substantially unaffected during processing of the therapeutic coating, i.e. low-density polyethylene (LDPE). In other embodiments, polymeric film 602 may comprise at least one of, for example, thermoplastic polyurethane (TPU), polytetrafluoroethylene (PTFE), polyethylene (including high-density polyethylene (HDPE)), polycaprolactone (PCL), silicone, and poly-lactic-acid (PLA), as well as other suitable polymers.

### EXAMPLES

### Example 1

Six low-density polyethylene (“LDPE”) film units were prepared, each film unit being a 1.5″×1.5″ square. As pretreatment procedure, a first and second film unit were left untreated; a third and fourth film unit were treated by heating the units to 80° C.; and a fifth and sixth film unit were treated via plasma-etching using plasma ignition, wherein the plasma pretreatment included a plasma power of 25 W, with an O₂ flow rate of 75 SCCM.

Each film unit was subjected to the N-TIPS coating method, performed by phase separation of meloxicam from dimethyl sulfoxide-benzyl alcohol (DMSO-Bn) co-solvent system by cooling at −20° C. using the TIPS method and extraction of DMSO-Bn with pH 2 acidic water using the NIPS method. More specifically, meloxicam was loaded onto each film unit by the following steps: (i) meloxicam was dissolved in a water-miscible DMSO-Bn co-solvent system at a meloxicam concentration of 30 mg/mL; (ii) each film unit was dipped into the drug solution; (iii) each film unit was placed in a cooling unit, the temperature was reduced to −20° C., and the film units remained in the cooling unit for 24 hours to induce phase separation via TIPS; (iv) each film unit was equilibrated to room temperature; (v) DMSO-Bn was extracted in pH 2 acidic water using the NIPS method, where each film unit was transferred to an acidic water bath for 20 minutes at room temperature, or about 20° C.; and (vi) each film unit was freeze-dried. During step (ii) of the above process, the first, third, and fifth film units were incubated at 80° C. for an hour after each film unit was dipped into the drug solution. During step (ii) of processing for the second, fourth, and sixth film units, each film unit remained at room temperature, or about 20° C.

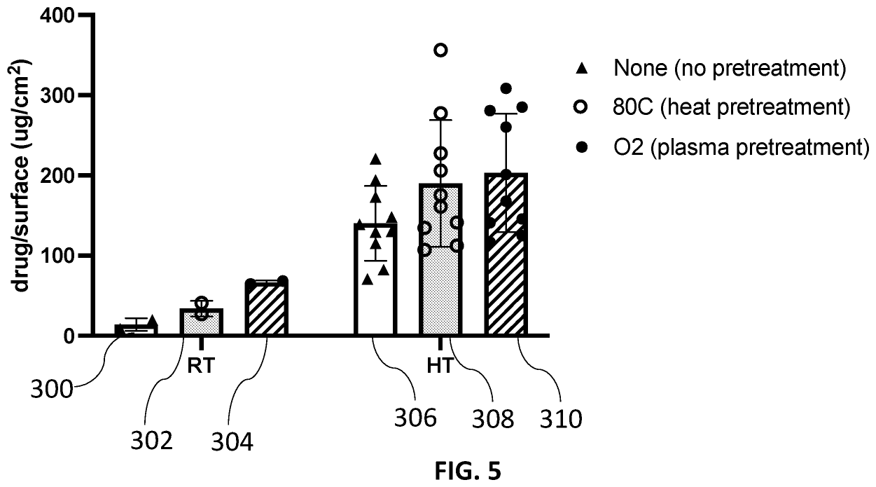

FIG. 5 illustrates the average drug loading of each film treatment type, wherein bar 300 illustrates the average drug loading for film units left untreated at the pretreatment stage and left at room temperature during the drug loading process, or step (ii) of the above method. As illustrated, the average drug loading amount was 13.9 μg/cm², with a standard deviation of +/−7.91 g/cm². Bar 302 illustrates the average drug loading for film units subjected to heated pretreatment, or pretreatment in which the corresponding films were heated to 80° C., and left at room temperature during the drug loading process, or step (ii) of the above method. As illustrated, the average drug loading amount was 33.7 μg/cm², with a standard deviation of +/−9.76 μg/cm². Bar 304 illustrates the average drug loading for film units subjected to plasma pretreatment and left at room temperature during the drug loading process, or step (ii) of the above method. As illustrated, the average drug loading amount was 66.4 μg/cm², with a standard deviation of +/−2.40 μg/cm². As illustrated, the average drug loading amount for film units left at room temperature during drug loading processing remained under 100 g/cm².

Bar 306 illustrates the average drug loading for film units left untreated at the pretreatment stage and heated to 80° C. during the drug loading process, or step (ii) of the above method. As illustrated, the average drug loading amount was 140.13 μg/cm², with a standard deviation of +/−7.91 μg/cm². Bar 308 illustrates the average drug loading for film units subjected to heated pretreatment, or pretreatment in which the corresponding films were heated to 80° C., and heated to 80° C. during the drug loading process, or step (ii) of the above method. As illustrated, the average drug loading amount was 189.85 μg/cm², with a standard deviation of +/−79.16 μg/cm². Bar 310 illustrates the average drug loading for film units subjected to plasma pretreatment and heated to 80° C. during the drug loading process, or step (ii) of the above method. As illustrated, the average drug loading amount was 189.85 μg/cm², with a standard deviation of +/−73.96 μg/cm². As illustrated, the average drug loading amount for film units heated to 80° C. during drug loading processing were from about 130 μg/cm² to about 205 μg/cm², where the standard deviation for pretreated film units reached nearly 300 μg/cm².

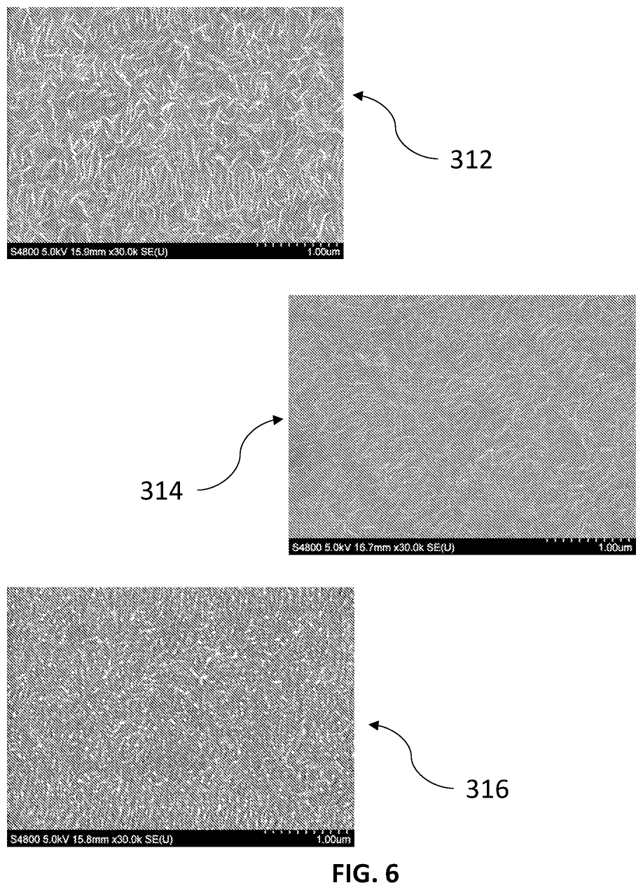

FIG. 6 provides a scanning electron microscopic view of a film unit subjected to each pretreatment method before drug loading of the film units. Image 312 provides a microscopic view of the microstructure of an untreated film unit. Image 314 provides a microscopic view of the microstructure of a film unit after heated pretreatment. Image 316 provides a microscopic view of the microstructure of a film unit after plasma-etching pretreatment.

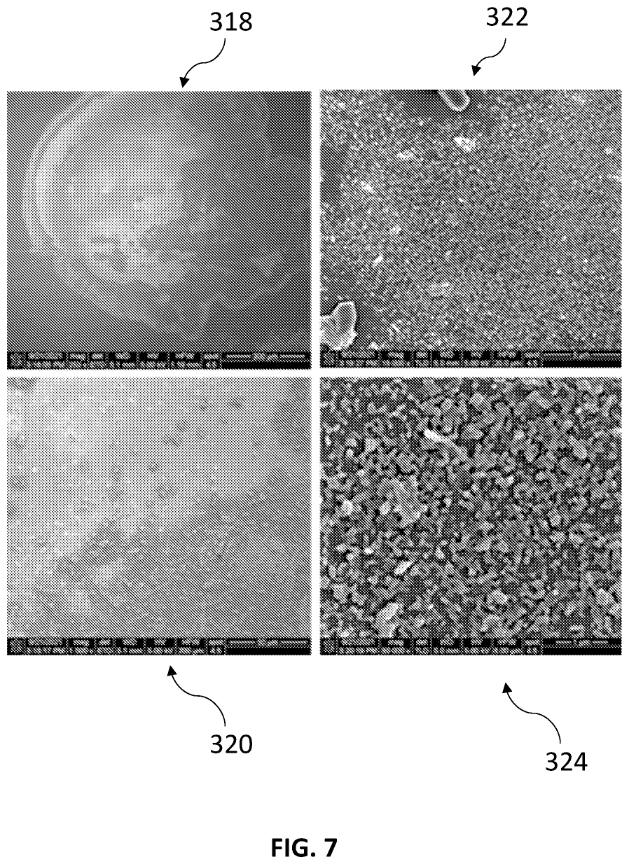

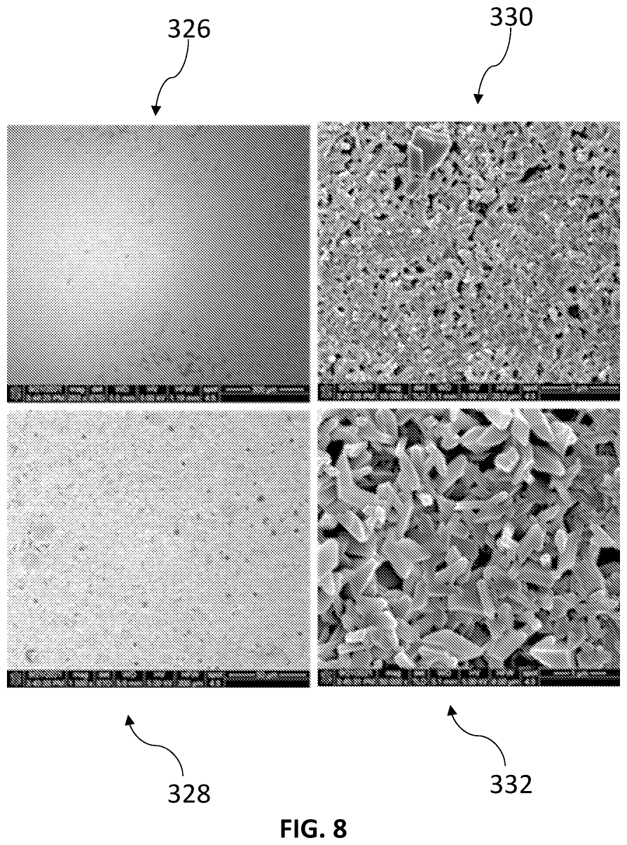

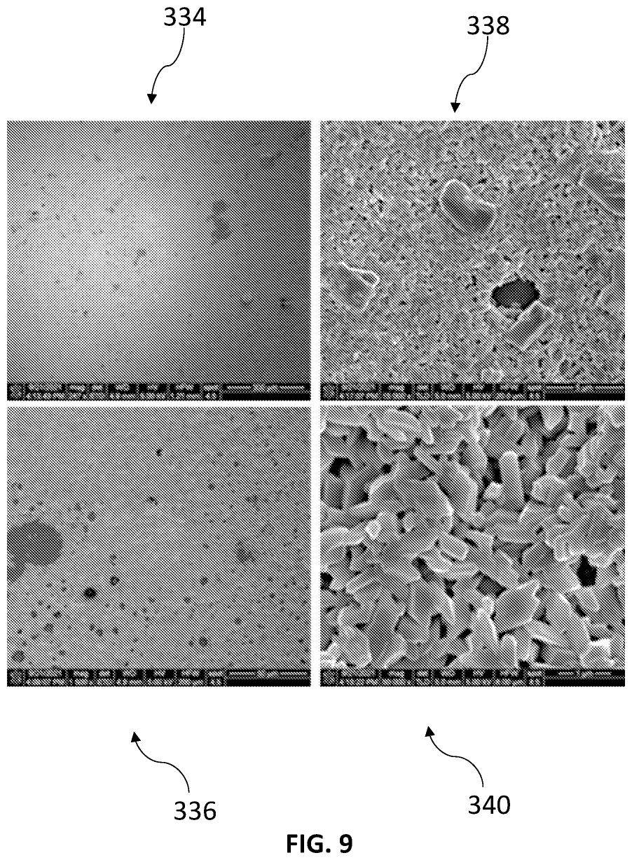

FIG. 7 provides increasingly magnified views of a drug loaded film unit post-N-TIPS method disclosed above, wherein the film unit was left untreated during the pretreatment phase and the film unit was heated to 80° C. during the drug loading process. Image 318 provides a view of said film unit at 253× magnification. Image 320 provides a view of said film unit at 1500× magnification. Image 322 provides a view of said film unit at 15,000× magnification. Image 324 provides a view of said film unit at 50,000× magnification. FIG. 8 provides increasingly magnified views of a drug loaded film unit post-N-TIPS method disclosed above, wherein the film unit was subjected to heated pretreatment during the pretreatment phase and the film unit was heated to 80° C. during the drug loading process. Image 326 provides a view of said film unit at 253× magnification. Image 328 provides a view of said film unit at 1500× magnification. Image 330 provides a view of said film unit at 15,000× magnification. Image 332 provides a view of said film unit at 50,000× magnification. FIG. 9 provides increasingly magnified views of a drug loaded film unit post-N-TIPS method disclosed above, wherein the film unit was subjected to plasma-etching pretreatment during the pretreatment phase and the film unit was heated to 80° C. during the drug loading process. Image 334 provides a view of said film unit at 253× magnification. Image 336 provides a view of said film unit at 1500× magnification. Image 338 provides a view of said film unit at 15,000× magnification. Image 340 provides a view of said film unit at 50,000× magnification.

### Example 2

Six low-density polyethylene (“LDPE”) tubes were prepared, each tube defining an inner lumen, wherein the diameter of the inner lumen, or the inner diameter, was about 0.038 cm and the diameter of the totality of the tube, or the outer diameter, was about 0.109 cm. The tubes were 10 cm in length. As pretreatment procedure, a first and second tube were left untreated; a third and fourth tube were treated by heating the tubes to 80° C.; and a fifth and sixth tube were treated via plasma-etching using plasma ignition, wherein the plasma pretreatment included a plasma power of 1.6, with an O₂ flow rate of 0.005 L/min, an H flow rate of 0.25 L/min, and a feed rate of 2 mm/min.

Each tube was subjected to the N-TIPS coating method, performed by phase separation of meloxicam from dimethyl sulfoxide-benzyl alcohol (DMSO-Bn) co-solvent system by cooling at −20° C. using the TIPS method and extraction of DMSO-Bn with pH 2 acidic water using the NIPS method. More specifically, meloxicam was loaded onto the inner surface of each tube by the following steps: (i) meloxicam was dissolved in a water-miscible DMSO-Bn co-solvent system at a meloxicam concentration of 30 mg/mL; (ii) the drug solution was introduced to each tube with a syringe, and each end of the tube was solder-sealed; (iii) each tube was placed in a cooling unit, the temperature was reduced to −20° C., and the tubes remained in the cooling unit for 24 hours to induce phase separation via TIPS; (iv) each film unit was equilibrated to room temperature; (v) DMSO-Bn was extracted in pH 2 acidic water using the NIPS method, where the acidic water was introduced to each tube by syringe at a rate of 12 μL/min for one hour at room temperature, or about 20° C.; and (vi) each tube was freeze-dried. During step (ii) of the above process, the first, third, and fifth tubes were incubated at 80° C. for an hour after each tube was filled with the drug solution. During step (ii) of processing for the second, fourth, and sixth tubes, each tube remained at room temperature, or about 20° C.

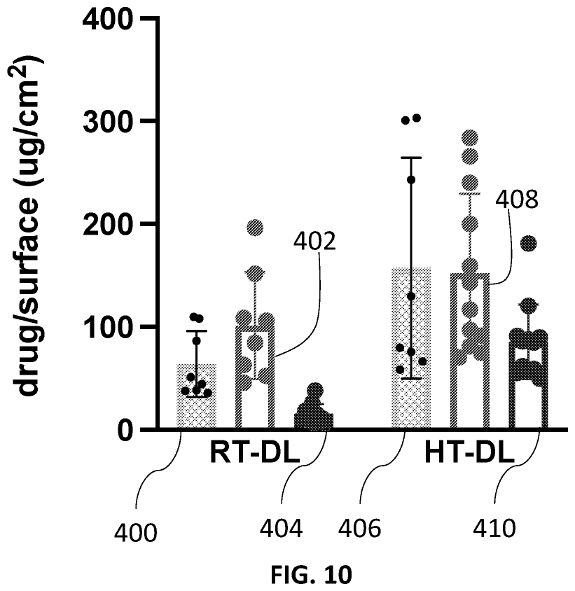

FIG. 10 illustrates the average drug loading of each tube treatment type, wherein bar 400 illustrates the average drug loading for tubes left untreated at the pretreatment stage and left at room temperature during the drug loading process, or step (ii) of the above method. As illustrated, the average drug loading amount was 64.05 μg/cm², with a standard deviation of +/−32.18 μg/cm². Bar 402 illustrates the average drug loading for tubes subjected to heated pretreatment, or pretreatment in which the corresponding tubes were heated to 80° C., and left at room temperature during the drug loading process, or step (ii) of the above method. As illustrated, the average drug loading amount was 101.14 μg/cm² with a standard deviation of +/−51.97 μg/cm². Bar 404 illustrates the average drug loading for tubes subjected to plasma pretreatment and left at room temperature during the drug loading process, or step (ii) of the above method. As illustrated, the average drug loading amount was 15.80 μg/cm² with a standard deviation of +/−9.41 μg/cm². As illustrated, the average drug loading amount for tubes left at room temperature during drug loading processing remained near 100 μg/cm², although the standard deviation for tubes subjected to heated pretreatment and room temperature drug loading reached over 150 μg/cm².

Bar 406 illustrates the average drug loading for tubes left untreated at the pretreatment stage and heated to 80° C. during the drug loading process, or step (ii) of the above method. As illustrated, the average drug loading amount was 157.19 μg/cm² with a standard deviation of +/−107.37 μg/cm². Bar 408 illustrates the average drug loading for tubes subjected to heated pretreatment, or pretreatment in which the corresponding tubes were heated to 80° C., and heated to 80° C. during the drug loading process, or step (ii) of the above method. As illustrated, the average drug loading amount was 152.02 μg/cm² with a standard deviation of +/−77.45 μg/cm². Bar 410 illustrates the average drug loading for tubes subjected to plasma pretreatment and heated to 80° C. during the drug loading process, or step (ii) of the above method. As illustrated, the average drug loading amount was 85.50 μg/cm² with a standard deviation of +/−36.42 μg/cm². As illustrated, the average drug loading amount for tubes heated to 80° C. during drug loading processing were from about 80 μg/cm² to about 160 μg/cm², where the standard deviation for untreated tubes reached nearly 275 μg/cm² and the standard deviation for heated pretreatment tubes reached nearly 250 μg/cm².

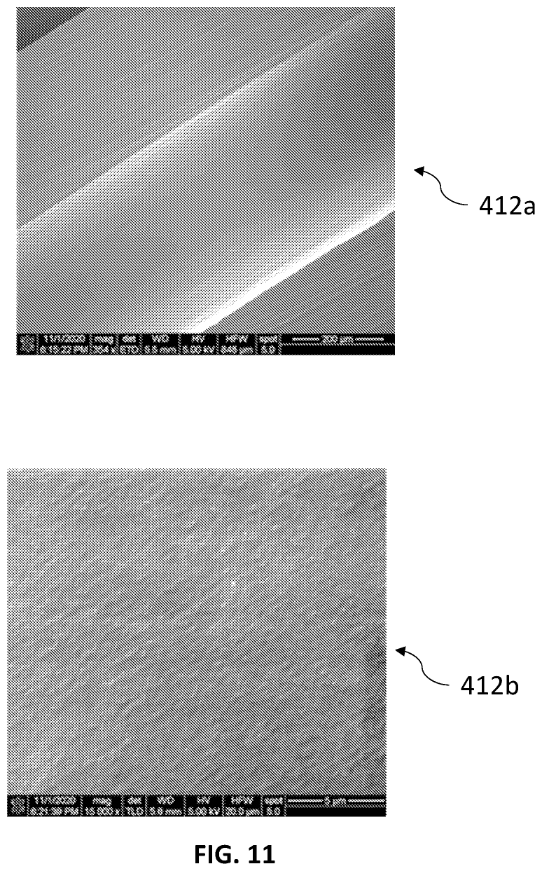

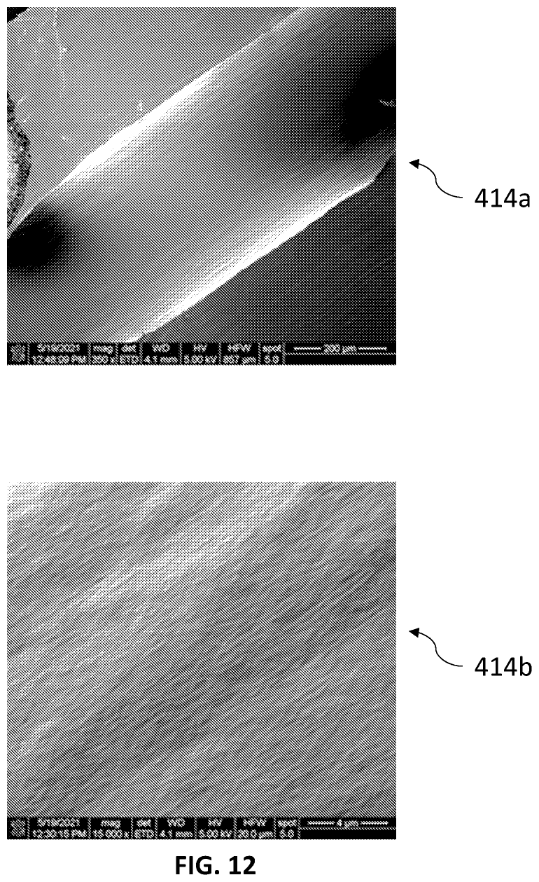

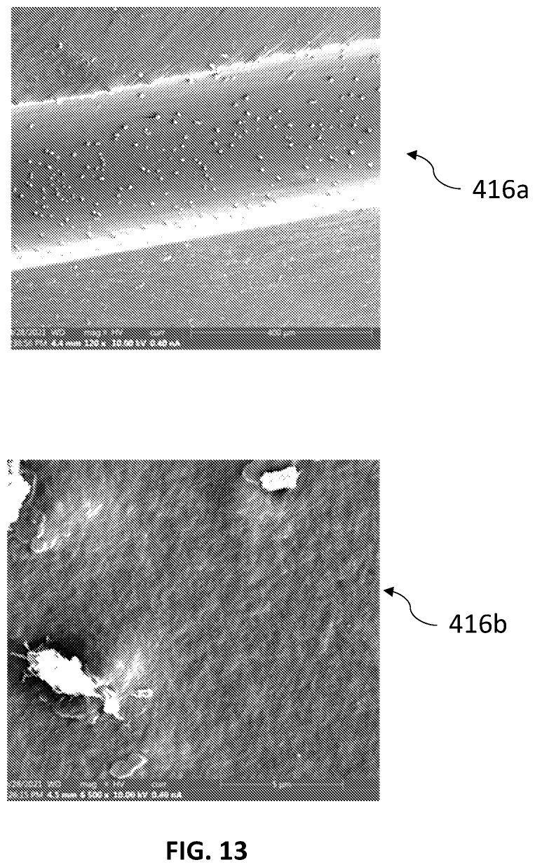

FIG. 11 provides a scanning electron microscopic view of the microstructure of a tube left untreated during the pretreatment phase before drug loading of the tube. Image 412a provides a 354× magnification microscopic view of the microstructure of the tube. Image 412b provides a 15,000× magnification microscopic view of the microstructure of the tube. FIG. 12 provides a scanning electron microscopic view of the microstructure of a tube subjected to heated pretreatment during the pretreatment phase before drug loading of the tube. Image 414a provides a 350× magnification microscopic view of the microstructure of the tube. Image 414b provides a 15,000× magnification microscopic view of the microstructure of the tube. FIG. 13 provides a scanning electron microscopic view of the microstructure of a tube subjected to plasma-etching during the pretreatment phase before drug loading of the tube. Image 416a provides a 120× magnification microscopic view of the microstructure of the tube. Image 416b provides a 6500× magnification microscopic view of the microstructure of the tube.

### Example 3

Low-density polyethylene (“LDPE”) tubes were prepared according to the method of Example 2 above, except that at step (ii) of the N-TIPS coating method, each tube was filled with the drug solution, solder sealed, and incubated at 80° C. for an hour.

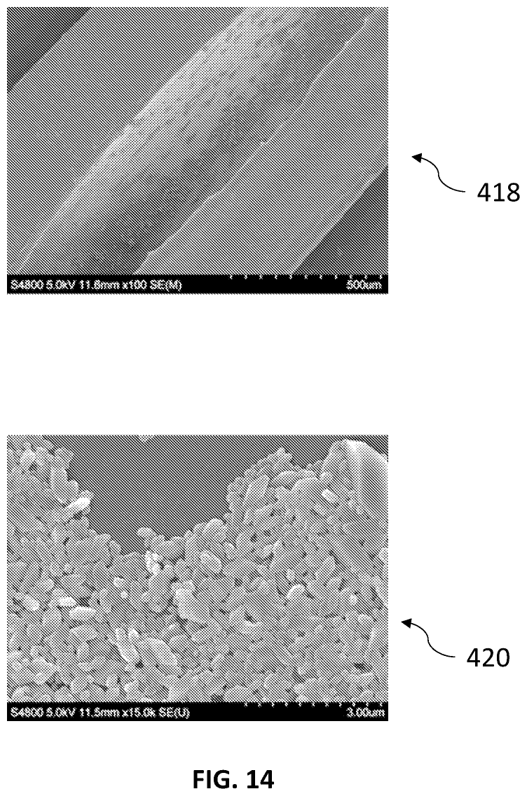

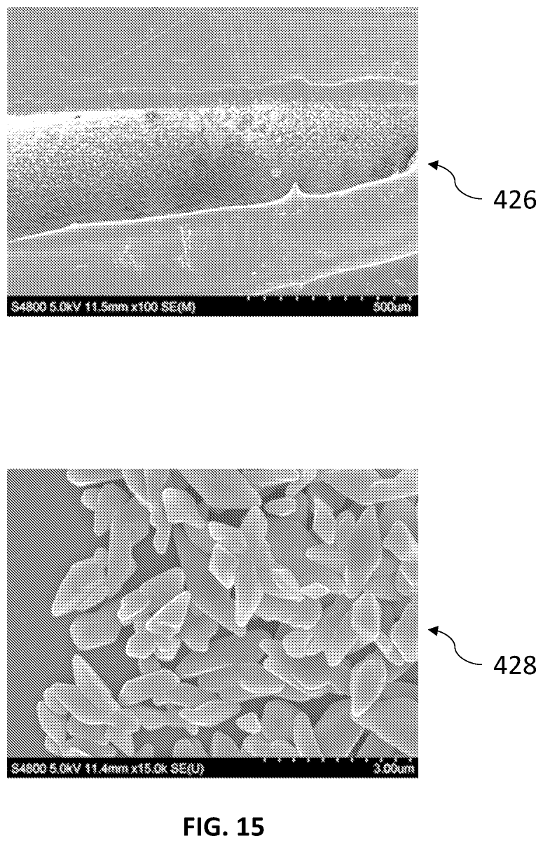

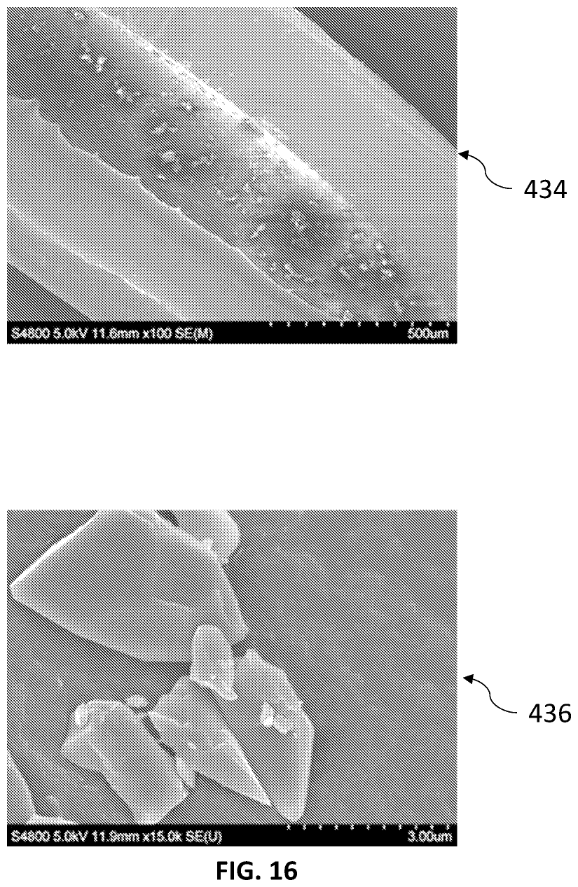

FIG. 14 provides increasingly magnified views of a drug loaded tube post-N-TIPS method disclosed above, wherein the film unit was left untreated during the pretreatment phase. Image 418 provides a view of said tube at 100× magnification. Image 420 provides a view of said tube at 15,000× magnification. FIG. 15 provides increasingly magnified views of a drug loaded tube post-N-TIPS method disclosed above, wherein the tube was subjected to heated pretreatment during the pretreatment phase. Image 426 provides a view of said tube at 100× magnification. Image 428 provides a view of said tube at 15,000× magnification. FIG. 16 provides increasingly magnified views of a drug loaded tube post-N-TIPS method disclosed above, wherein the tube was subjected to plasma-etching pretreatment during the pretreatment phase. Image 434 provides a view of said film unit at 100× magnification. Image 436 provides a view of said tube at 15,000× magnification.

### Example 4

Low-density polyethylene (“LDPE”) tubes were prepared, each tube defining an inner lumen, wherein the length of each tube was 10 cm and the volume of each tube was 0.0113 cm³. As pretreatment procedure, a first tube was left untreated; a second tube was treated by heating the tube to 80° C.; and a third tube was treated via plasma-etching using plasma ignition, wherein the plasma pretreatment included a plasma power of 1.6, with an O₂ flow rate of 0.005 L/min, an H flow rate of 0.25 L/min, and a feed rate of 2 mm/min. The tubes were then subjected to an N-TIPS procedure as described above in relation to Example 3.

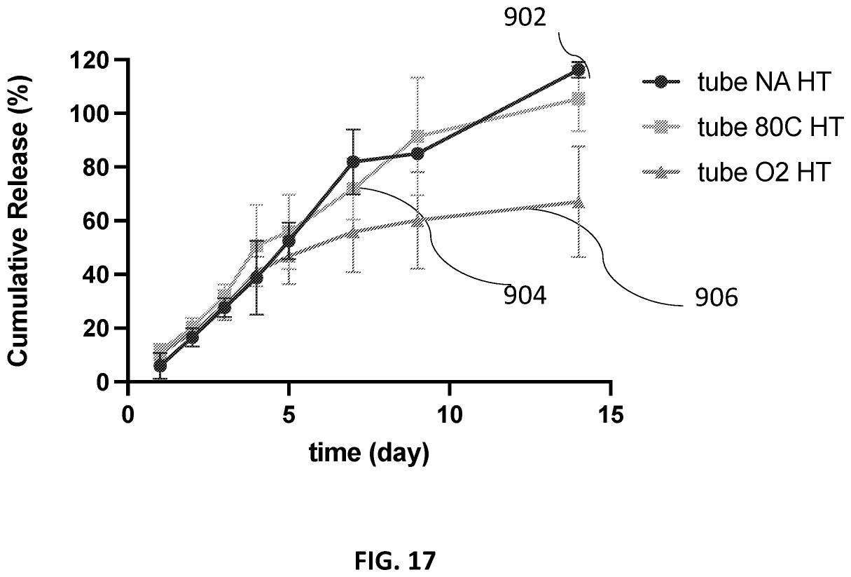

Release media in the form of 0.9% saline was introduced to each tube for a 14-day period, and the cumulative release % of drug release over the 14-day period was recorded. The sampled media was mixed with DMSO. The results are provided in FIG. 17, where release line 902 corresponds with the average cumulative release % of nontreated tubes, release line 904 corresponds with the average cumulative release % of tubes subjected to heated pretreatment, and release line 906 corresponds with the average cumulative release % of tubes subjected to plasma-etching pretreatment. Tubes subjected to the plasma-etching pretreatment reached about 70% cumulative release of meloxicam over the 14-day elution period. Tubes which were left untreated during the pretreatment phase reached nearly 100% cumulative release of meloxicam over the 14-day elution period. Tubes subjected to the heated pretreatment also reached about 100% cumulative release of meloxicam over the 14-day elution period.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A method of coating a medical device with a therapeutic agent, the method comprising:
introducing a solution comprising the therapeutic agent to a surface of the medical device;
freezing the medical device with the solution so that the therapeutic agent precipitates from the solution; and
washing the medical device with a non-solvent, wherein the precipitated therapeutic agent remains as a coating on the surface of the medical device.

2. The method of claim 1, wherein the surface is an inner surface of an inner lumen of a line set tubing.

3. The method of claim 2, wherein the line set tubing is a component of an infusion device.

4. The method of claim 1, wherein the surface is an inner surface of an inner lumen of a catheter.

5. The method of claim 1, wherein the surface is a surface of a film.

6. The method of claim 1, wherein the surface is a polymeric surface.

7. The method of claim 6, wherein the surface comprises low-density polyethylene.

8. The method of claim 1, further comprising the step of pretreating the surface of the medical device with at least one of a heated pretreatment or a plasma-etching pretreatment before introducing the solution comprising the therapeutic agent to the surface.

9. The method of claim 1, further comprising the step of heating the medical device with the solution to a temperature of about 80° C. after introducing the solution to the surface.

10. The method of claim 1, wherein during the freezing step, the therapeutic agent crystallizes on the surface.

11. The method of claim 1, wherein only the therapeutic agent precipitates.

12. The method of claim 11, wherein the therapeutic agent is water-insoluble.

13. The method of claim 1, wherein the therapeutic agent is meloxicam.

14. The method of claim 1, wherein a matrix comprising the therapeutic agent and a polymer precipitates.

15. The method of claim 14, wherein the therapeutic agent is water-soluble.

16. An infusion device for extended wear, the infusion device comprising:
a base;
an adhesive configured to couple the base to a skin of a patient; and
a catheter configured to pierce the skin of the patient, wherein the catheter is in fluid communication with a tubing including a therapeutic coating on an inner surface of an inner lumen defined by the tubing, the therapeutic coating disposed on the inner surface of the inner lumen defined by the tubing using N-TIPS.

17. The infusion device of claim 16, wherein the therapeutic coating consists essentially of a therapeutic agent.

18. The infusion device of claim 17, wherein the therapeutic agent is meloxicam.

19. The infusion device of claim 16, wherein the catheter is configured to remain inserted in the patient for 14 days, and wherein the therapeutic coating is configured to continuously release the therapeutic agent while the catheter is inserted.

20. The infusion device of claim 16, wherein the therapeutic agent is in a crystalline form.

21. The infusion device of claim 16, wherein the tubing is comprised of low-density polyethylene.
